(19) United States
(12) Patent Application Publication
(10) Pub. No.: US 2026/0124067 A1
(43) Pub. Date: May 7, 2026

(54) Oral Positioning Device

(71) Applicant: Metal Industries Research & Development Centre, Kaohsiung City (TW)

(72) Inventors: TUNG-LIN TSAI, Tainan City (TW); Chun-Chieh Tseng, Kaohsiung City (TW); Yu-Sheng Lin, Kaohsiung City (TW); Li-Wen Weng, Kaohsiung City (TW); Bang-Sia Chen, Kinmen County (TW); Yin-Tzu Chen, Tainan City (TW); Yue-Jun Wang, New Taipei City (TW); Pei-Hua Wang, Kaohsiung City (TW)

(21) Appl. No.: 18/935,693

(22) Filed: Nov. 4, 2024

(51) Int. Cl. A61F 5/56

(52) U.S. Cl. CPC A61F 5/566

(57) ABSTRACT

An oral positioning device is provided to solve the problem of surgical risks associated with the conventional method of improving sleep apnea. The oral positioning device includes an upper positioning cover, a lower positioning cover, and two positioning members respectively connected to the upper positioning cover and the lower positioning cover. The upper positioning cover includes a groove, and an outer sidewall and an inner sidewall are respectively located on two opposite sides of the groove of the upper positioning cover. The lower positioning cover includes a groove, and an outer sidewall and an inner sidewall are respectively located on two opposite sides of the groove of the lower positioning cover. The lower positioning cover protrudes beyond the upper positioning cover by a protruding distance. The oral positioning device can prevent the user from breathing obstruction caused by tongue base muscles during sleep.

### BACKGROUND OF THE INVENTION

### 1. Field of the Invention

The present invention relates to a medical device and, more particularly, to an oral positioning device for fixing the positions of upper and lower jaws.

### 2. Description of the Related Art

The major causes of sleep apnea include a narrow airway which is inherent or caused by obesity, or collapse of the tongue base muscles resulting from insufficient muscle tension in the airway. Thus, the airway is blocked, resulting in breathing obstruction. A patient suffering from sleep apnea snores and, thus, adversely affects the sleep quality of the patient and the person sleeping beside the patient. Therefore, the patient may suffer from drowsiness in the daytime and reduced concentration and, in severe cases, may even cause sudden death due to apnea during the sleep period.

A currently available solution for treating sleep apnea is removing a portion of muscles through surgery. However, infection is apt to occur due to the surgery.

Thus, it is necessary to improve the conventional methods for improving sleep apnea.

### SUMMARY OF THE INVENTION

To solve the above problems, it is an objective of the present invention to provide an oral positioning device through a non-surgical approach to avoid airway blockage caused by the tongue base muscles.

As used herein, the term “a”, “an” or “one” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

As used herein, the term “engagement”, “coupling”, “assembly”, or similar terms is used to include separation of connected members without destroying the members after connection or inseparable connection of the members after connection. A person having ordinary skill in the art would be able to select according to desired demands in the material or assembly of the members to be connected.

An oral positioning device according to the present invention includes an upper positioning cover, a lower positioning cover, and two positioning members respectively connected to the upper positioning cover and the lower positioning cover. The upper positioning cover includes a groove, and an outer sidewall and an inner sidewall are respectively located on two opposite sides of the groove of the upper positioning cover. The lower positioning cover includes a groove, and an outer sidewall and an inner sidewall are respectively located on two opposite sides of the groove of the lower positioning cover. The lower positioning cover protrudes beyond the upper positioning cover by a protruding distance.

Thus, in the oral positioning device according to the present invention, by providing the upper positioning cover and the lower positioning cover respectively disposed on the upper row of teeth and the lower row of teeth of the user, the two positioning members may position the upper positioning cover and the lower positioning cover, such that the lower positioning cover protrudes beyond the upper positioning cover in the protruding direction. Thus, the lower jaw of the user protrudes beyond the upper jaw of the user, such that the tongue base muscles behind the lower jaw are remote from the airway. Therefore, by fixing the oral positioning device in the oral cavity of the user, the user is prevented from breathing obstruction caused by the tongue base muscles during sleep, thereby avoiding airway blockage resulting in breathing obstruction.

In an example, the two positioning members are respectively connected to the outer sidewall of the upper positioning cover and the outer sidewall of the lower positioning cover. Therefore, the two positioning members can fix the upper positioning cover and the lower positioning cover to provide a positioning effect.

In an example, the two positioning members are respectively connected to the inner sidewall of the upper positioning cover and the inner sidewall of the lower positioning cover. Therefore, the two positioning members can fix the upper positioning cover and the lower positioning cover to provide a positioning effect.

In an example, each of the two positioning members includes a first connecting section and a second connecting section. The first connecting section is connected to the upper positioning cover. The second connecting section is connected to the lower positioning cover. Therefore, a predetermined spacing is maintained between the upper positioning cover and the lower positioning cover.

In an example, the first connecting section includes a coupling hole coupled with a peg on the outer sidewall of the upper positioning cover. The second connecting section includes a coupling hole coupled with a peg on the outer sidewall of the lower positioning cover. Therefore, a predetermined spacing is maintained between the upper positioning cover and the lower positioning cover.

In an example, each of the two positioning members further includes an elastic portion interconnected between the first connecting section and the second connecting section, wherein the first connecting section and the second connecting section move toward each other when the elastic portion deforms elastically, and wherein the first connecting section and the second connecting section move away from each other under an elastic returning force of the elastic portion. Therefore, wear caused by mutual contact between the upper positioning cover and the lower positioning cover resulting from frequent biting of upper and lower rows of teeth of the user can be avoided.

In an example, the upper positioning cover and the lower positioning cover have a symmetric central line in a protruding direction, and wherein the outer sidewall of the lower positioning cover on the symmetric central line is spaced from the outer sidewall of the upper positioning cover by the protruding distance along the protruding direction. Therefore, the lower jaw of the user may be spaced from the upper jaw by the protruding distance.

In an example, the protruding distance is 3-5 mm. Therefore, the tongue base muscles behind the lower jaw may be remote from the airway to prevent collapse of the tongue base muscles while the user is sleeping, thereby avoiding breathing obstruction caused by airway blockage.

In an example, the oral positioning device further comprises a tongue pressing member disposed on the upper positioning cover or the lower positioning cover. Therefore, the tongue pressing member may restrict the tongue at the lower jaw, thereby avoiding the tongue from collapsing toward the airway.

In an example, the tongue pressing member includes a plurality of flow guiding holes. Therefore, the plurality of flow guiding holes may permit outflow of saliva secreted by the tongue.

In an example, the tongue pressing member is disposed on the upper positioning cover and includes a limiting portion. The tongue pressing member protrudes toward the lower positioning cover, such that the limiting portion is located in an area surrounded by the lower positioning cover. Therefore, the tongue pressing member may restrict the tongue at the lower jaw, thereby avoiding the tongue from collapsing toward the airway.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 an exploded, perspective view of an oral positioning device of a first embodiment according to the present invention.

FIG. 2 is a perspective view of the oral positioning device of the first embodiment according to the present invention after assembly.

FIG. 3 is a top elevational view of the oral positioning device of the first embodiment according to the present invention.

FIG. 4 is a schematic view illustrating use of the oral positioning device of the first embodiment according to the present invention.

FIG. 5 an exploded, perspective view of an oral positioning device of a second embodiment according to the present invention.

FIG. 6 is a perspective view of the oral positioning device of the second embodiment according to the present invention after assembly.

FIG. 7 is a top elevational view of the oral positioning device of the second embodiment according to the present invention.

When the terms “front”, “rear”, “left”, “right”, “up”, “down”, “top”, “bottom”, “inner”, “outer”, “side”, and similar terms are used herein, it should be understood that these terms have reference only to the structure shown in the drawings as it would appear to a person viewing the drawings and are utilized only to facilitate describing the invention, rather than restricting the invention.

### DETAILED DESCRIPTION OF THE INVENTION

In order to make the above and other objectives, features, and advantages of the present invention clearer and easier to understand, preferred embodiments of the present invention will be described hereinafter in connection with the accompanying drawings. Furthermore, the elements designated by the same reference numeral in various figures will be deemed as identical, and the description thereof will be omitted.

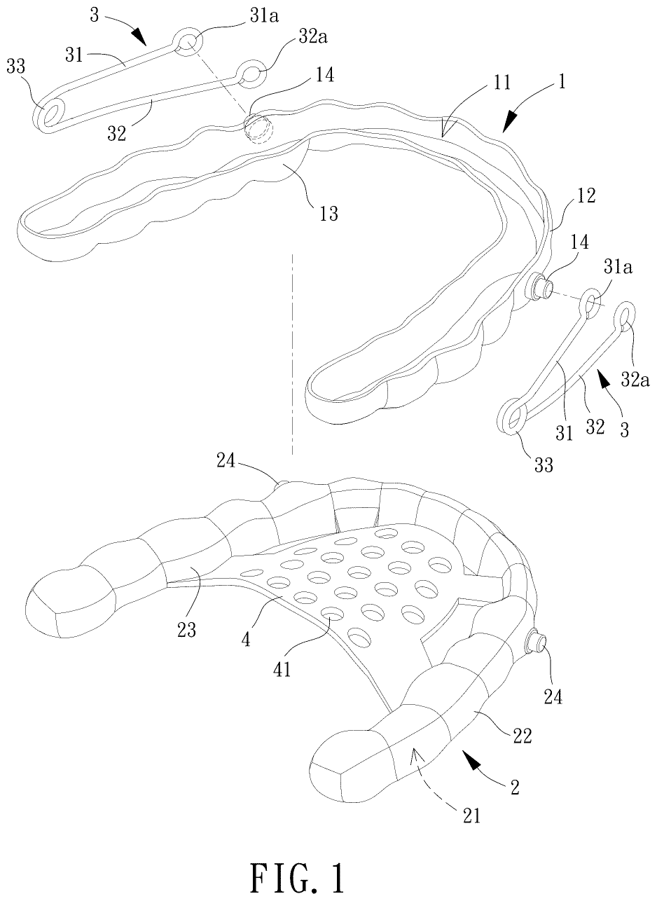

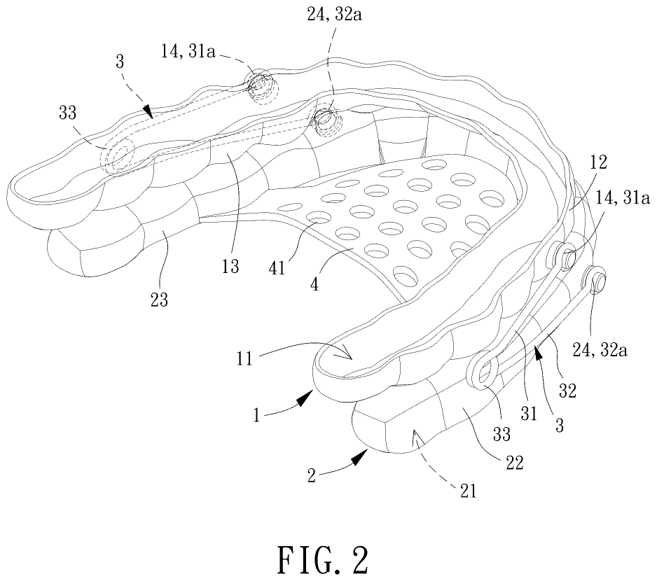

With reference to FIGS. 1 and 2, an oral positioning device of a first embodiment according to the present invention the present invention includes an upper positioning cover 1, a lower positioning cover 2, and two positioning members 3. The upper positioning cover 1 and the lower positioning cover 2 are connected by the two positioning members 3.

The upper positioning cover 1 may be fixed in an oral cavity of a user and is configured to position the upper jaw of the user. Specifically, the upper positioning cover 1 may be an upper mouth guard. The upper positioning cover 1 may be made of thermoplastic elastomer (TPE) to provide excellent abrasion resistance and flexibility, such that the upper positioning cover 1 may be easily fixed to the upper row of teeth of the user. The upper positioning cover 1 may include a groove 11, such that the upper positioning cover 1 may be disposed on the upper row of teeth of the user through the groove 11. Preferably, the groove 11 may have a shape corresponding to the profile of the upper row of teeth of the user. For example, a molding process may be conducted on the upper row of teeth to form the upper positioning cover 1.

Furthermore, the upper positioning cover 1 may include an outer sidewall 12 and an inner sidewall 13 respectively on two opposite sides of the groove 11. Each of the outer sidewall 12 and the inner sidewall 13 may be arcuate, such that the upper positioning cover 1 may have an outline corresponding to the upper row of teeth of the user. The outer sidewall 12 faces the mouth of the user. The inner sidewall 13 faces the oral cavity of the user. Thus, the upper positioning cover 1 may be securely disposed on the upper row of teeth of the user.

The lower positioning cover 2 is configured to position the lower jaw of the user. The lower positioning cover 2 may be a lower mouth guard. The lower positioning cover 2 may be made of thermoplastic elastomer (TPE). The lower positioning cover 2 may include a groove 21, such that the lower positioning cover 2 may be disposed on the lower row of teeth of the user through the groove 21. Preferably, the groove 21 may have a shape corresponding to the profile of the lower row of teeth of the user. Furthermore, the lower positioning cover 2 may include an outer sidewall 22 and an inner sidewall 23 respectively on two opposite sides of the groove 21. Each of the outer sidewall 22 and the inner sidewall 23 may be arcuate, such that the lower positioning cover 2 may have an outline corresponding to the lower row of teeth of the user. The outer sidewall 22 faces the lips of the user. The inner sidewall 23 faces the oral cavity of the user. Thus, the lower positioning cover 2 may be securely disposed on the lower row of teeth of the user.

The two positioning members 3 are respectively connected to the upper positioning cover 1 and the lower positioning cover 2 to position the upper positioning cover 1 and the lower positioning cover 2, such that the upper positioning cover 1 and the lower positioning cover 2 may face each other in a vertical direction in the oral cavity of the user. The two positioning members 3 may be connected to the upper positioning cover 1 and the lower positioning cover 2 through adhering, coupling or integral formation. Specifically, the two positioning members 3 may be connected to the outer sidewall 12 of the upper positioning cover 1 and the outer sidewall 22 of the lower positioning cover 2, respectively. Alternatively, the two positioning members 3 may be connected to the inner sidewall 13 of the upper positioning cover 1 and the inner sidewall 23 of the lower positioning cover 2.

In this embodiment, each of the two positioning members 3 may include a first connecting section 31 and a second connecting section 32. The first connecting section 31 is connected to the upper positioning cover 1. For example, the first connecting section 31 includes a coupling hole 31a which may be coupled with a peg 14 on the outer sidewall 12 of the upper positioning cover 1. The second connecting section 32 is connected to the lower positioning cover 2. The second connecting section 32 includes a coupling hole 32a which may be coupled with a peg 24 on the outer sidewall 22 of the lower positioning cover 2. An elastic portion 33 may be disposed between the first connecting section 31 and the second connecting section 32. The first connecting section 31 and the second connecting section 32 move toward each other when the elastic portion 33 deforms elastically. The first connecting section 31 and the second connecting section 32 move away from each other under an elastic returning force of the elastic portion 33. Thus, a predetermined spacing is maintained between the upper positioning cover 1 and the lower positioning cover 2 due to the returning elasticity of the elastic portion 33. Therefore, wear caused by mutual contact between the upper positioning cover 1 and the lower positioning cover 2 resulting from frequent biting of upper and lower rows of teeth of the user can be avoided.

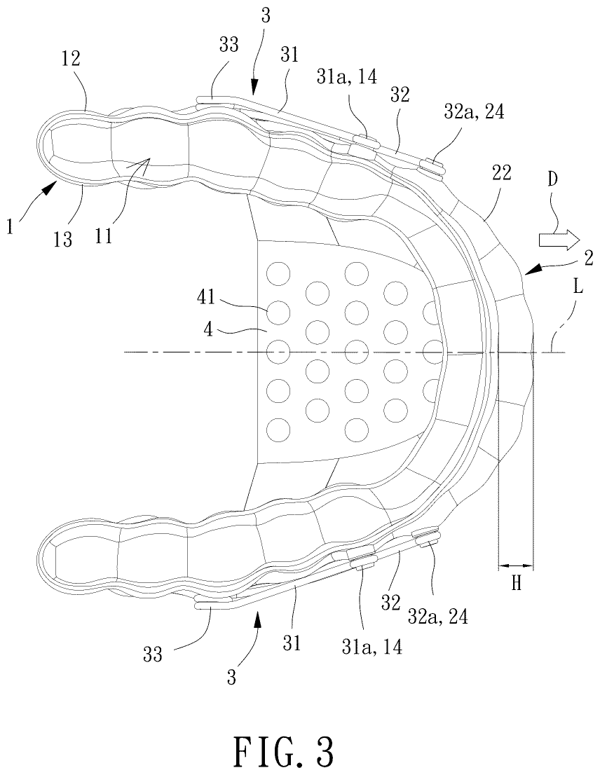

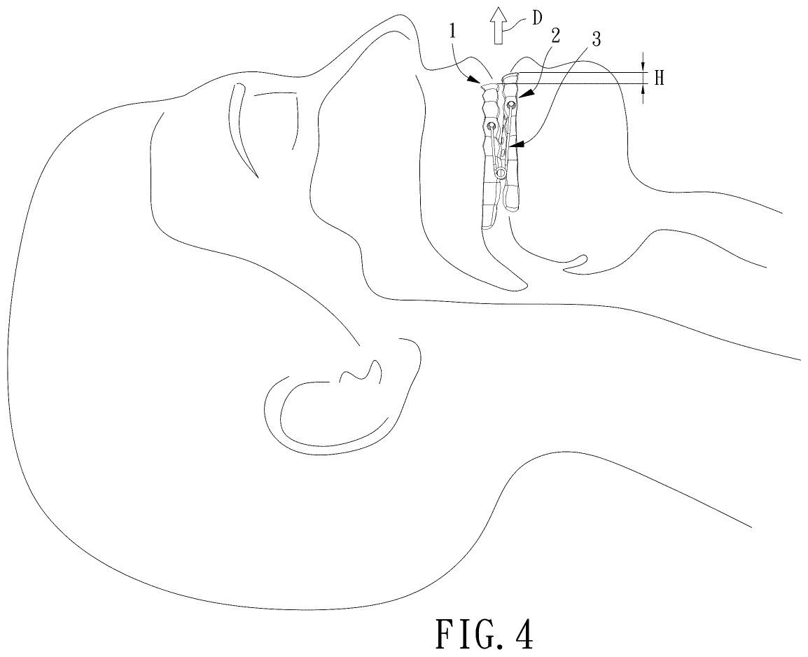

With reference to FIGS. 3 and 4, it should be noted that the two positioning members 3 may position the upper positioning cover 1 and the lower positioning cover 2 in a manner that the upper positioning cover 1 and the lower positioning cover 2 are misaligned. Specifically, as viewed from an inner side of the oral cavity of the user toward an outer side of the oral cavity of the user, a protruding direction D is defined from the inner sidewalls 13 and 23 of the upper positioning cover 1 and the lower positioning cover 2 toward the outer sidewalls 12 and 22. The lower positioning cover 2 protrudes beyond the upper positioning cover 1 in the protruding direction D. Furthermore, the upper positioning cover 1 and the lower positioning cover 2 have a symmetrical central line L in the protruding direction D. The outer sidewall 22 of the lower positioning cover 2 on the symmetric central line L is spaced from the outer sidewall 12 of the upper positioning cover 1 by the protruding distance H along the protruding direction D. The protruding distance H may be 3-5 mm. Thus, after the upper positioning cover 1 and the lower positioning cover 2 are respectively disposed on the upper and lower rows of teeth of the user, the lower jaw of the user may protrude beyond the lower jaw of the user by the protruding distance H. Therefore, the tongue base muscles behind the lower jaw are remote from the airway to prevent collapse of the tongue base muscles while the user is sleeping, thereby avoiding breathing obstruction caused by airway blockage.

With reference to FIGS. 1 and 2, the oral positioning device according to the present invention may further include a tongue pressing member 4 which may be located on the lower positioning cover 2. The tongue pressing member 4 may be in the form of a plate. For example, a periphery of the tongue pressing member 4 may be connected to the inner sidewall 23 of the lower positioning cover 2, such that the tongue pressing member 4 may be aligned with the tongue, such that the tongue pressing member 4 may restrict the tongue at the lower jaw, thereby avoiding the tongue from collapsing toward the airway. The tongue pressing member 4 may include a plurality of flow guiding holes 41 which may be in the form of through-holes. The saliva secreted by the tongue may flow outward via the plurality of flow guiding holes 41.

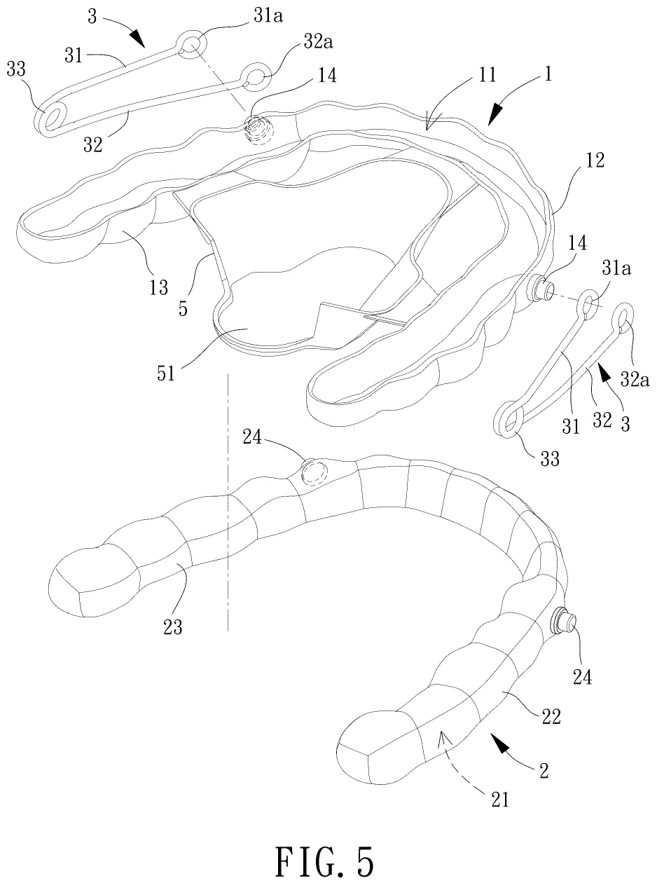

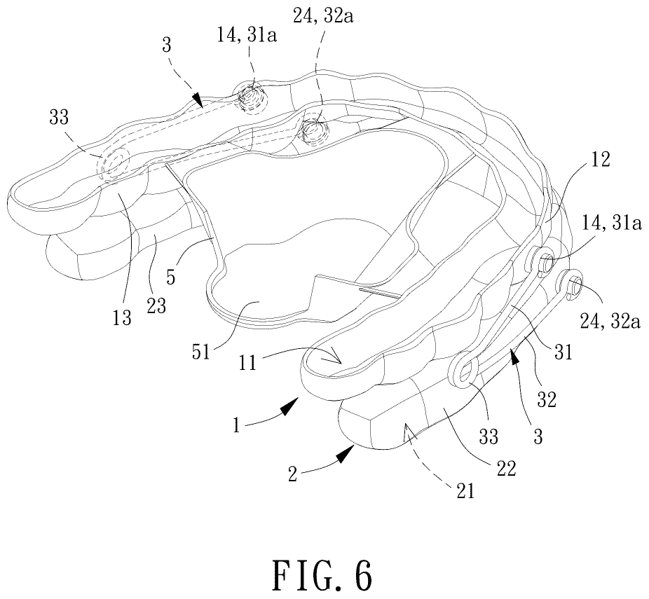

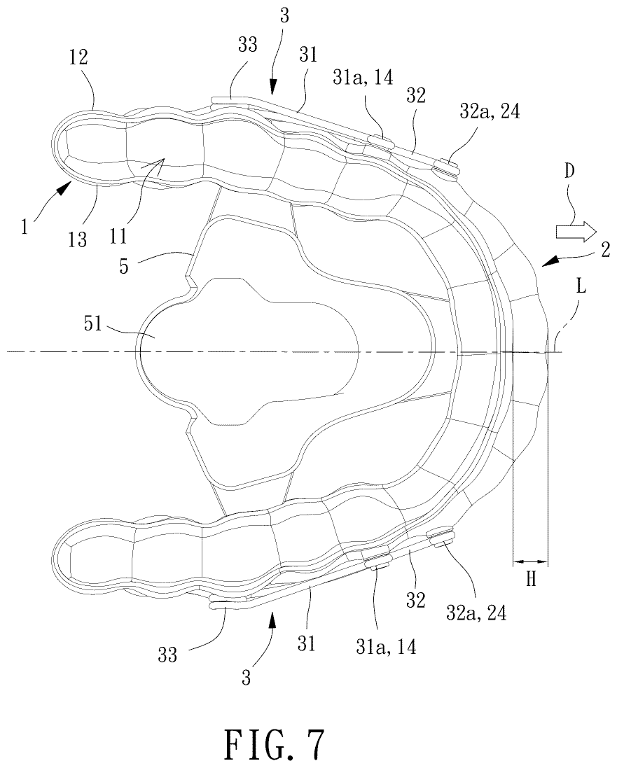

FIGS. 5-7 show an oral positioning device of a second embodiment according to the present invention. This embodiment is substantially the same as the first embodiment mentioned above. In this embodiment, the tongue pressing member 5 is located on the upper positioning cover 1. A periphery of the tongue pressing member 5 may be connected to the inner sidewall 13 of the upper positioning cover 1. The tongue pressing member 5 includes a limiting portion 51. The tongue pressing member 5 protrudes toward the lower positioning cover 2, such that the limiting portion 51 is located in an area surrounded by the lower positioning cover 2. Therefore, the tongue pressing member 5 may restrict the tongue at the lower jaw, thereby avoiding the tongue from collapsing toward the airway.

In summary, in the oral positioning device according to the present invention, by providing the upper positioning cover and the lower positioning cover respectively disposed on the upper row of teeth and the lower row of teeth of the user, the two positioning members may position the upper positioning cover and the lower positioning cover, such that the lower positioning cover protrudes beyond the upper positioning cover in the protruding direction. Thus, the lower jaw of the user protrudes beyond the upper jaw of the user, such that the tongue base muscles behind the lower jaw are remote from the airway. Therefore, by fixing the oral positioning device in the oral cavity of the user, the user is prevented from breathing obstruction caused by the tongue base muscles during sleep, thereby avoiding airway blockage resulting in breathing obstruction.

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims.

What is claimed is:

1. An oral positioning device comprising:
an upper positioning cover including a groove, wherein an outer sidewall and an inner sidewall are respectively located on two opposite sides of the groove of the upper positioning cover;
a lower positioning cover including a groove, wherein an outer sidewall and an inner sidewall are respectively located on two opposite sides of the groove of the lower positioning cover; and
two positioning members respectively connected to the upper positioning cover and the lower positioning cover, wherein the lower positioning cover protrudes beyond the upper positioning cover by a protruding distance.

2. The oral positioning device as claimed in claim 1, wherein the two positioning members are respectively connected to the outer sidewall of the upper positioning cover and the outer sidewall of the lower positioning cover.

3. The oral positioning device as claimed in claim 1, wherein the two positioning members are respectively connected to the inner sidewall of the upper positioning cover and the inner sidewall of the lower positioning cover.

4. The oral positioning device as claimed in claim 1, wherein each of the two positioning members includes a first connecting section and a second connecting section, wherein the first connecting section is connected to the upper positioning cover, and wherein the second connecting section is connected to the lower positioning cover.

5. The oral positioning device as claimed in claim 4, wherein the first connecting section includes a coupling hole coupled with a peg on the outer sidewall of the upper positioning cover, and wherein the second connecting section includes a coupling hole coupled with a peg on the outer sidewall of the lower positioning cover.

6. The oral positioning device as claimed in claim 4, wherein each of the two positioning members further includes an elastic portion interconnected between the first connecting section and the second connecting section, wherein the first connecting section and the second connecting section move toward each other when the elastic portion deforms elastically, and wherein the first connecting section and the second connecting section move away from each other under an elastic returning force of the elastic portion.

7. The oral positioning device as claimed in claim 1, wherein the upper positioning cover and the lower positioning cover have a symmetric central line in a protruding direction, and wherein the outer sidewall of the lower positioning cover on the symmetric central line is spaced from the outer sidewall of the upper positioning cover by the protruding distance along the protruding direction.

8. The oral positioning device as claimed in claim 1, wherein the protruding distance is 3-5 mm.

9. The oral positioning device as claimed in claim 1, further comprising a tongue pressing member disposed on the upper positioning cover or the lower positioning cover.

10. The oral positioning device as claimed in claim 9, wherein the tongue pressing member includes a plurality of flow guiding holes.

11. The oral positioning device as claimed in claim 9, wherein the tongue pressing member is disposed on the upper positioning cover and includes a limiting portion, and wherein the tongue pressing member protrudes toward the lower positioning cover, such that the limiting portion is located in an area surrounded by the lower positioning cover.
